(19) United States
(12) Patent Application Publication
(10) Pub. No.: US 2021/0147238 A1
(43) Pub. Date: May 20, 2021

(54) TRANSFER MATERIAL LAYERS FOR GRAPHENE FABRICATION PROCESS

(71) Applicants: REGENTS OF THE UNIVERSITY OF MINNESOTA, Minneapolis, MN (US); Boston Scientific Scimed, Inc., Maple Grove, MN (US)

(72) Inventors: Steven J. Koester, Edina, MN (US); Xue Zhen, Minneapolis, MN (US); Philippe Pierre Joseph Buhlmann, Minneapolis, MN (US); Qun Su, Minneapolis, MN (US); Justin Theodore Nelson, Minneapolis, MN (US); Gregory J. Sherwood, North Oaks, MN (US)

(21) Appl. No.: 17/096,104

(22) Filed: Nov. 12, 2020

Related U.S. Application Data
(60) Provisional application No. 62/935,941, filed on Nov. 15, 2019.

(51) Int. Cl. C01B 32/19; C09D 5/00; C09D 9/00; C09D 127/12; G01N 33/00; G01N 27/22

(52) U.S. Cl. CPC C01B 32/19; C09D 5/008; C09D 9/005; C01B 2204/22; G01N 33/0027; G01N 27/227; C09D 127/12

(57) ABSTRACT

Embodiments herein relate to methods and systems for applying a transfer material layer to graphene during a graphene fabrication process. In an embodiment, a method of producing a graphene sensor element is included. The method includes forming a graphene layer on a growth substrate and applying a fluoropolymer coating layer over the graphene layer. The method includes removing the growth substrate and transferring the graphene and fluoropolymer coating layers onto a transfer substrate, where the graphene layer is disposed on the transfer substrate and the fluoropolymer layer is disposed on the graphene layer. The method also includes removing the fluoropolymer coating layer. Other embodiments are also included herein.

This application claims the benefit of U.S. Provisional Application No. 62/935,941 filed Nov. 15, 2019, the content of which is herein incorporated by reference in its entirety.

### FIELD

Embodiments herein relate to methods and systems for applying a transfer material layer to graphene during a graphene fabrication process. More specifically, embodiments herein relate to methods and systems including the use of a fluoropolymer as a transfer material layer for graphene during a graphene fabrication process.

### BACKGROUND

Graphene is a form of carbon containing a single layer of carbon atoms in a hexagonal lattice. Graphene has a high strength and stability due to its tightly packed sp² hybridized orbitals, where each carbon atom forms one sigma (σ) bond each with its three neighboring carbon atoms and has one p orbital projected out of the hexagonal plane. The p orbitals of the hexagonal lattice can hybridize to form a π band suitable for non-covalent interactions with both electron rich or electron deficient molecules.

During a graphene manufacturing process, a single layer of graphene can be transferred from a metal growth substrate onto a different substrate. However, the transfer process may result in an undesired residue on the graphene surface and discontinuous coverage of the substrate upon which the single graphene layer is disposed after the transfer.

### SUMMARY

In a first aspect, a method of producing a graphene sensor element is included. The method can include forming a graphene layer on a growth substrate, applying a fluoropolymer coating layer over the graphene layer, removing the growth substrate, transferring the graphene and fluoropolymer coating layers onto a transfer substrate, where the graphene layer is disposed on the transfer substrate and the fluoropolymer layer is disposed on the graphene layer. The method can include removing the fluoropolymer coating layer.

In a second aspect, in addition to one or more of the preceding or following aspects, or in the alternative to some aspects, the growth substrate can include copper.

In a third aspect, in addition to one or more of the preceding or following aspects, or in the alternative to some aspects, the fluoropolymer can include poly[4,5-difluoro-2,2-bis(trifluoromethyl)-1,3-dioxole-co-tetrafluoroethylene], or derivatives thereof.

In a fourth aspect, in addition to one or more of the preceding or following aspects, or in the alternative to some aspects, where a mole ratio of dioxole to tetrafluoroethylene is from 1:99 to 99:1.

In a fifth aspect, in addition to one or more of the preceding or following aspects, or in the alternative to some aspects, the fluoropolymer can include poly[oxy(1,1,2,2,3,3-hexafluoro-1,2-propanediyl)], poly[oxy(1,1,2,2,3,3-hexafluoro-1,3-propanediyl)], or derivatives thereof.

In a sixth aspect, in addition to one or more of the preceding or following aspects, or in the alternative to some aspects, where the fluoropolymer has a solubility in a solvent of greater than 0.1 wt. %.

In a seventh aspect, in addition to one or more of the preceding or following aspects, or in the alternative to some aspects, where applying a fluoropolymer includes a spin coating process, an ink-jet printing, a spray coating process, or a chemical vapor deposition process.

In an eighth aspect, in addition to one or more of the preceding or following aspects, or in the alternative to some aspects, where removing the growth substrate includes applying a ferric chloride solution or an ammonium persulfate solution.

In a ninth aspect, in addition to one or more of the preceding or following aspects, or in the alternative to some aspects, where the fluoropolymer coating layer is at least about 10 nanometers thick.

In a tenth aspect, in addition to one or more of the preceding or following aspects, or in the alternative to some aspects, further can include sterilizing the graphene and fluoropolymer coating layers disposed on the transfer substrate before removing the fluoropolymer coating layer.

In an eleventh aspect, in addition to one or more of the preceding or following aspects, or in the alternative to some aspects, where removing the fluoropolymer coating layer includes applying a solvent can include a perfluoroalkane, a partially fluorinated alkane, a partially fluorinated haloalkane, a perfluorinated mono- or polycyclic alkane, a perfluorinated singly or multiply alkyl substituted mono- or polycyclic alkane, a perfluoroaromatic, a (perfluoroalkyl)benzene, a perfluoroether, a perfluorodiether, a perfluorotriether, a perfluoroalkyl alkyl ether, a perfluoro(trialkylamine), or mixtures of two or more of any preceding solvents.

In a twelfth aspect, in addition to one or more of the preceding or following aspects, or in the alternative to some aspects, where removing the fluoropolymer coating layer is performed immediately before use of the graphene sensor element to analyze a gas sample.

In a thirteenth aspect, a method of producing a graphene sensor element is included, the method forming a graphene layer on a growth substrate, functionalizing the graphene layer, applying a fluoropolymer coating layer over the graphene layer, removing the growth substrate, transferring the graphene and fluoropolymer coating layers onto a transfer substrate, and removing the fluoropolymer coating layer.

In a fourteenth aspect, in addition to one or more of the preceding or following aspects, or in the alternative to some aspects, the growth substrate can include copper.

In a fifteenth aspect, in addition to one or more of the preceding or following aspects, or in the alternative to some aspects, the fluoropolymer coating layer can include one or more fluoropolymers can include perfluoropolymers and perfluoropolyethers.

In a sixteenth aspect, in addition to one or more of the preceding or following aspects, or in the alternative to some aspects, the fluoropolymer can include poly[4,5-difluoro-2,2-bis(trifluoromethyl)-1,3-dioxole-co-tetrafluoroethylene], poly[oxy(1,1,2,2,3,3-hexafluoro-1,2-propanediyl)], or poly[oxy(1,1,2,2,3,3-hexafluoro-1,3-propanediyl)], or derivatives thereof.

In a seventeenth aspect, in addition to one or more of the preceding or following aspects, or in the alternative to some aspects, where removing the growth substrate includes applying a ferric chloride solution or an ammonium persulfate solution.

In an eighteenth aspect, in addition to one or more of the preceding or following aspects, or in the alternative to some aspects, further can include sterilizing the graphene sensor element before removing the fluoropolymer coating layer.

In a nineteenth aspect, in addition to one or more of the preceding or following aspects, or in the alternative to some aspects, where removing the fluoropolymer coating layer includes applying a solvent can include a perfluoroalkane, a partially fluorinated alkane, a partially fluorinated haloalkane, a perfluorinated mono- or polycyclic alkane, a perfluorinated singly or multiply alkyl substituted mono- or polycyclic alkane, a perfluoroaromatic, a (perfluoroalkyl)benzene, a perfluoroether, a perfluorodiether, a perfluorotriether, a perfluoroalkyl alkyl ether, a perfluoro(trialkylamine), or mixtures of two or more of any preceding solvents.

In a twentieth aspect, in addition to one or more of the preceding or following aspects, or in the alternative to some aspects, where removing the fluoropolymer coating layer is performed immediately before use of the graphene sensor element to analyze a gas sample.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic perspective view of a graphene assembly during different operations of a method in accordance with various embodiments herein.

FIG. 2 is a schematic cross-sectional view of a graphene assembly during different operations of a method along line 2-2′ of FIG. 1 in accordance with various embodiments herein.

FIG. 3 is a schematic perspective view of an additional graphene assembly during different operations of a method in accordance with various embodiments herein.

FIG. 4 is a schematic cross-sectional view of an additional graphene assembly during different operations of a method along line 3-3′ of FIG. 3 in accordance with various embodiments herein.

FIG. 5 is a schematic perspective view of a graphene varactor in accordance with various embodiments herein

FIG. 6 is a schematic cross-sectional view of a portion of a graphene varactor in accordance with various embodiments herein.

FIG. 7 is a schematic block diagram of circuitry to measure the capacitance of a plurality of graphene sensors in accordance with various embodiments herein.

FIG. 8 shows atomic force microscopy (AFM) images of a graphene surface in accordance with various embodiments herein.

FIG. 9 shows atomic force microscopy (AFM) images of a graphene surface in accordance with various embodiments herein.

FIG. 10 shows x-ray photoelectron spectroscopy (XPS) images of a graphene surface in accordance with various embodiments herein.

FIG. 11 shows atomic force microscopy (AFM) images and optical microscopy images of various graphene surfaces in accordance with various embodiments herein.

FIG. 12 shows atomic force microscopy (AFM) images and optical microscopy images of various graphene surfaces in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

As referenced above, a single layer of graphene can be transferred from a metal growth substrate onto a different substrate during a manufacturing process. In some cases, a layer of a transfer material (or transfer support layer) can be disposed onto the graphene temporarily to provide support to the graphene layer as the growth substrate is removed and the graphene is transferred to the different substrate. However, the transfer process may result in an undesired residue of the transfer material on the graphene surface and thus can result in discontinuous coverage of the single graphene layer on the substrate upon which it is disposed after the transfer.

However, embodiments herein specifically include the use of a fluoropolymer coating layer as a transfer material layer for graphene during a graphene fabrication process. The fluoropolymers used herein are unique in terms of the narrow range of compounds that serve effective solvents to them. This allows for the precise and thorough removal of the fluoropolymer without damage to the graphene layer or any compounds used to functionalize the surface thereof. Thus, in embodiments herein, graphene monolayers can be transferred from growth substrate to a different substrate by specifically utilizing a fluoropolymer layer as a transfer material layer for graphene grown by chemical vapor deposition (CVD), or similar methods.

The fluoropolymer layer can be used as a transfer material layer during a graphene transfer process as well as a protection layer during fabrication processes and for storage. Spin-coating, ink-jet printing, spray coating process, chemical vapor deposition, including plasma-enhanced chemical vapor deposition, or similar method of deposition of a fluoropolymer solution in a fluorous solvent onto a graphene layer produces a uniform fluorocarbon layer that can be directly used without curing. In various embodiments, a plasma vapor deposition process can include use of hexafluoropropylene (i.e., C₃F₆) as a precursor to fluoropolymer layer formation. In some embodiments.

It will be appreciated that in various embodiments, the transfer material layer can include a plasticized fluoropolymer layer. In various embodiments, the fluoropolymer can be mixed with a fluorous plasticizer. Fluorous plasticizers suitable for use herein can include, but not be limited to, one or more of a linear perfluorocarbon, a branched perfluorocarbon, a monocyclic perfluorocarbon, a polycyclic perfluorocarbon, a perfluoroether, a perfluoropolyether, a perfluoroamine, a perfluoropolyamine, and the like.

The coated fluoropolymer layer provides sufficient mechanical strength and flexibility to hold delaminated graphene intact before being disposed onto a target substrate, such as a transfer substrate described further herein. The transfer processes can be performed in a water bath, where any trapped water underneath the graphene layer can be removed by spin-drying and/or vacuum bake-out, with the fluoropolymer layer remaining on the graphene.

When used as a protection layer, the fluoropolymer layer on the graphene layer effectively protects it from mechanical scratches and chemical contamination. The fluoropolymer layer can be removed by dissolving it in fluorous solvents, including with or without mechanical agitation and heating. The removal process leaves minimal residue or deformation on the graphene surface. In addition, the coating and removal of the fluoropolymer layer does not damage any covalent or non-covalent functionalization on the graphene layer. It therefore can be used to transfer already functionalized graphene and prevent possible chemical degradation of the surface functionalization groups on graphene.

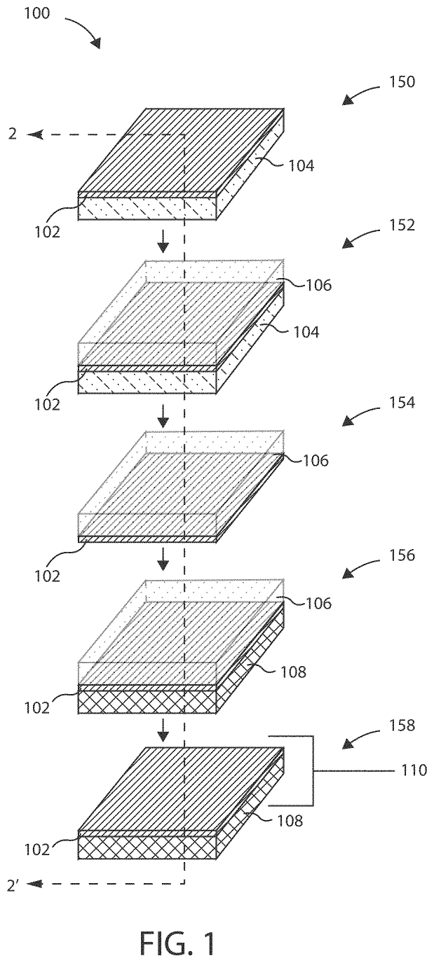

Referring now to FIG. 1, a schematic perspective view of a graphene assembly during a method 100 of producing a graphene sensor element is shown in accordance with various embodiments herein. The method 100 includes forming a graphene layer 102 on a growth substrate 104 at operation 150. In various embodiments, the step of forming a graphene layer 102 on a growth substrate 104 can include using a chemical vapor deposition process, as will be discussed further below. In various embodiments, the growth substrate 104 can include copper or copper oxide.

The method 100 includes applying a fluoropolymer coating layer 106 over the graphene layer 102 at operation 152. In various embodiments, the fluoropolymer coating layer 106 can include one or more fluoropolymers, including, but not to be limited to, perfluoropolymers and perfluoropolyethers. In various embodiments, the perfluoropolymers include amorphous perfluoropolymers. Fluoropolymers suitable for use in the methods herein are described further below. In various embodiments, applying a fluoropolymer can include a spin coating process. In other embodiments, applying a fluoropolymer can include an ink jet printing, spray coating process, chemical vapor deposition, including plasma-enhanced chemical vapor deposition, or similar method of deposition. In various embodiments, a plasma vapor deposition process can include use of hexafluoropropylene (i.e., C₃F₆) as a precursor to fluoropolymer layer formation.

The method 100 includes removing the growth substrate 104 at operation 154, leaving the fluoropolymer coating layer 106 disposed on the graphene layer 102. In various embodiments, removing the growth substrate 104 can include etching the growth substrate 104 using an etchant. In some embodiments, the etchant can include, but is not limited to, ammonium persulfate ((NH₄)₂S₂O₈) or ferric chloride (Fe(III)Cl₃) solutions.

It will be appreciated that in various embodiments, various persulfates are suitable for use herein, including potassium persulfate (K₂S₂O₈), sodium persulfate(Na₂S₂O₈), or any persulfate solution having the formula MS₂O₈, where M is any inert counter ion. It will be appreciated that in various embodiments, various ferric compounds are suitable for use herein, including ferric sulfate (Fe(III)₂(SO₄)₃), ferric nitrate ((Fe(III)(NO₃)₃), or any ferric solution having the formula MFe(III), where M is any inert counter ion.

The method 100 includes transferring the graphene layer 102 with the fluoropolymer coating layer 106 disposed thereon onto a transfer substrate 108 at operation 156. In some embodiments the transfer substrate 108 can include silicon (Si) or silicon dioxide (SiO₂), however other materials are also contemplated herein. The method 100 includes removing the fluoropolymer coating layer 106 at operation 158, leaving the graphene sensor element 110, including a graphene layer 102 disposed on the surface of a transfer substrate 108. In various embodiments, the step of removing the fluoropolymer coating layer is performed immediately before use of the graphene sensor element to analyze a gas sample. In various embodiments, the method 100 further can include sterilizing the graphene assembly, including the graphene and fluoropolymer coating layers disposed on the transfer substrate, as obtained at operation 156, before the step of removing the fluoropolymer coating layer. In some embodiments, the transfer substrate can include a dielectric material, as will be discussed in more detail below.

In various embodiments, removing the fluoropolymer coating layer 106 can include dissolving the fluoropolymer coating layer 106 using a fluorous solvent. In various embodiments, the step of removing the fluoropolymer coating layer using a fluorous solvent can include applying a fluorous solvent including, but not to be limited to a perfluoroalkane, a partially fluorinated alkane, a partially fluorinated haloalkane, a perfluorinated mono- or polycyclic alkane, a perfluorinated singly or multiply alkyl substituted mono- or polycyclic alkane, a perfluoroaromatic, a (perfluoroalkyl)benzene, a perfluoroether, a perfluorodiether, a perfluorotriether, a perfluoroalkyl alkyl ether, a perfluoro(trialkylamine), or mixtures of two or more of any preceding solvents. Suitable fluorous solvents for use in the methods herein are discussed further below.

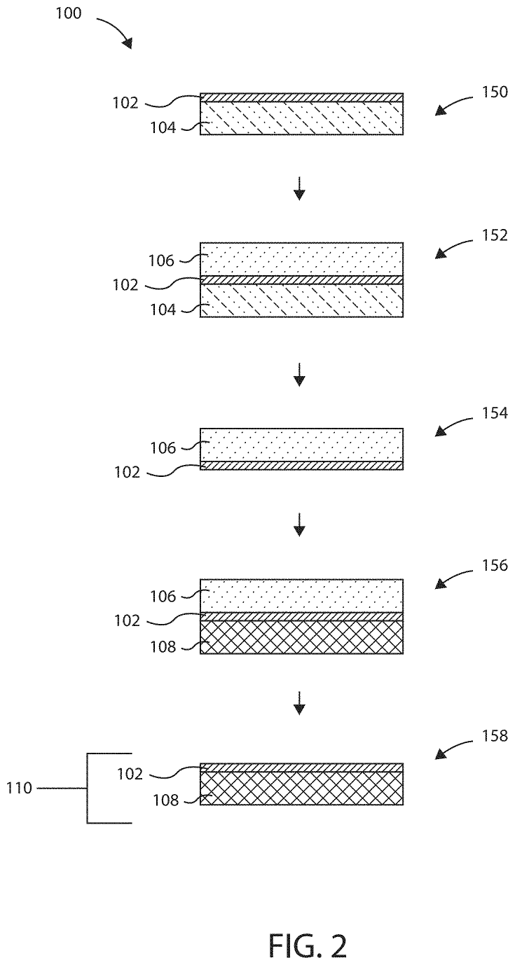

Referring now to FIG. 2, a schematic cross-sectional view of a graphene assembly during a method along line 2-2′ of FIG. 1 is shown in accordance with various embodiments herein. The method 100 includes forming a graphene layer 102 on a growth substrate 104 at operation 150. The method 100 includes applying a fluoropolymer coating layer 106 over the graphene layer 102 at operation 152. The method 100 includes removing the growth substrate 104 at operation 154, leaving the fluoropolymer coating layer 106 disposed on the graphene layer 102. The method 100 includes transferring the graphene layer 102 with the fluoropolymer coating layer 106 disposed thereon onto a transfer substrate 108 at operation 156. The method 100 includes removing the fluoropolymer coating layer 106 at operation 158, leaving the graphene sensor element 110, including a graphene layer 102 disposed on the surface of a transfer substrate 108. In various embodiments, the method 100 further can include sterilizing the graphene assembly, including the graphene and fluoropolymer coating layers disposed on the transfer substrate, as obtained at operation 156, before the step of removing the fluoropolymer coating layer.

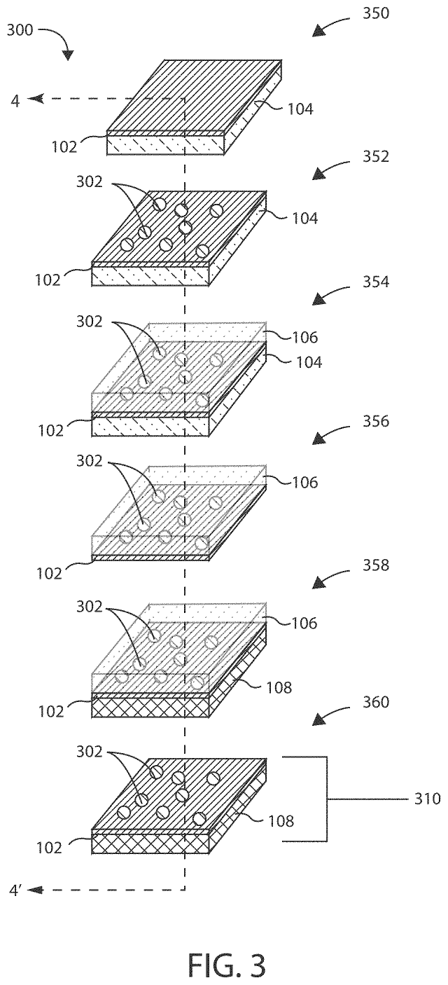

Referring now to FIG. 3, a schematic perspective view of a graphene assembly during a method 300 of producing a graphene sensor element is shown in accordance with various embodiments herein. The method 300 includes forming a graphene layer 102 on a growth substrate 104 at operation 350. In various embodiments, the step of forming a graphene layer 102 on a growth substrate 104 can include using a chemical vapor deposition process, as will be discussed further below. In various embodiments, the growth substrate 104 can include copper or copper oxide. The method 300 includes functionalizing the graphene layer at operation 352 with one or more functional groups 302. Various functional groups suitable for use herein are discussed further below.

The method 300 includes applying a fluoropolymer coating layer 106 over the graphene layer 102 functionalized with functional groups 302 at operation 354. In various embodiments, the fluoropolymer coating layer 106 can include one or more fluoropolymers, including, but not to be limited to, perfluoropolymers and perfluoropolyethers. In various embodiments, the perfluoropolymers include amorphous perfluoropolymers. Fluoropolymers suitable for use in the methods herein are described further below. In various embodiments, applying a fluoropolymer can include a spin coating process. In other embodiments, applying a fluoropolymer can include an ink jet printing, spray coating process, chemical vapor deposition, including plasma-enhanced chemical vapor deposition, or similar method of deposition. In various embodiments, a plasma vapor deposition process can include use of hexafluoropropylene (i.e., C₃F₆) as a precursor to fluoropolymer layer formation.

The method 300 includes removing the growth substrate 104 at operation 356, leaving the fluoropolymer coating layer 106 disposed on the graphene layer 102 functionalized with functional groups 302. In various embodiments, removing the growth substrate 104 can include etching the growth substrate 104 using an etchant. In some embodiments, the etchant can include, but is not limited to, ammonium persulfate ((NH₄)₂S₂O₈) or ferric chloride (Fe(III)Cl₃) solutions.

It will be appreciated that in various embodiments, various persulfates are suitable for use herein, including potassium persulfate (K₂S₂O₈), sodium persulfate(Na₂S₂O₈), or any persulfate solution having the formula MS₂O₈, where M is any inert counter ion. It will be appreciated that in various embodiments, various ferric compounds are suitable for use herein, including ferric sulfate (Fe(III)₂(SO₄)₃), ferric nitrate ((Fe(III)(NO₃)₃), or any ferric solution having the formula MFe(III), where M is any inert counter ion.

The method 300 includes transferring the graphene layer 102 functionalized with functional groups 302 with the fluoropolymer coating layer 106 disposed thereon onto a transfer substrate 108 at operation 358. In some embodiments the transfer substrate 108 can include silicon (Si) or silicon dioxide (SiO₂). The method 300 includes removing the fluoropolymer coating layer 106 at operation 360, leaving the functionalized graphene sensor element 310, including a graphene layer 102 functionalized with functional groups 302 disposed on the surface of a transfer substrate 108. In various embodiments, the step of removing the fluoropolymer coating layer is performed immediately before use of the graphene sensor element to analyze a gas sample. In various embodiments, the method 300 further can include sterilizing the graphene assembly, including the graphene functionalized with functional groups and fluoropolymer coating layers disposed on the transfer substrate, as obtained at operation 358, before the step of removing the fluoropolymer coating layer. In some embodiments, the transfer substrate can include a dielectric material, as will be discussed in more detail below.

In various embodiments, removing the fluoropolymer coating layer 106 can include dissolving the fluoropolymer coating layer 106 using a fluorous solvent. In various embodiments, the step of removing the fluoropolymer coating layer using a fluorous solvent can include applying a fluorous solvent, including, but not to be limited to a perfluoroalkane, a partially fluorinated alkane, a partially fluorinated haloalkane, a perfluorinated mono- or polycyclic alkane, a perfluorinated singly or multiply alkyl substituted mono- or polycyclic alkane, a perfluoroaromatic, a (perfluoroalkyl)benzene, a perfluoroether, a perfluorodiether, a perfluorotriether, a perfluoroalkyl alkyl ether, a perfluoro(trialkylamine), or mixtures of two or more of any preceding solvents. Suitable fluorous solvents for use in the methods herein are discussed further below.

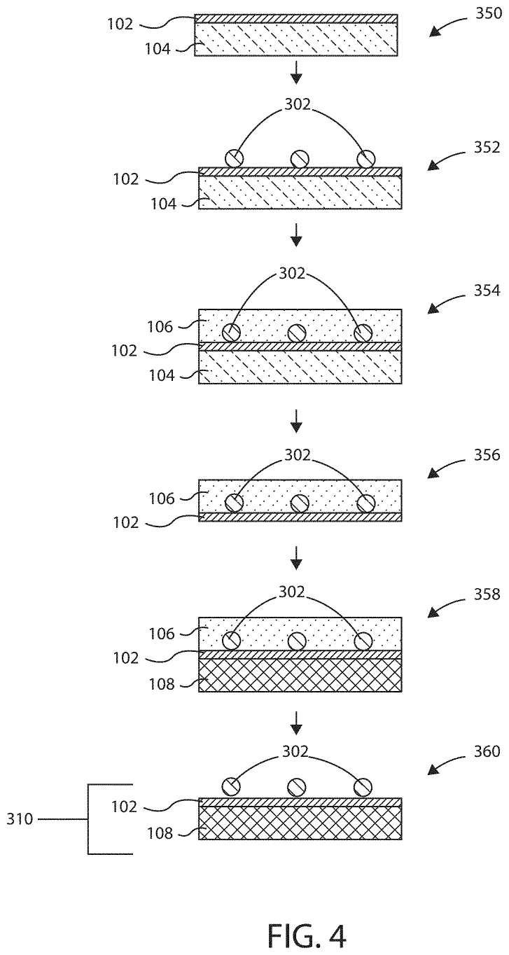

Referring now to FIG. 4, a schematic cross-sectional view of a graphene assembly during a method along line 4-4′ of FIG. 3 is shown in accordance with various embodiments herein. The method 300 includes forming a graphene layer 102 on a growth substrate 104 at operation 350. The method 300 includes functionalizing the graphene layer at operation 352 with one or more functional groups 302. The method 300 includes applying a fluoropolymer coating layer 106 over the graphene layer 102 functionalized with functional groups 302 at operation 354. The method 300 includes removing the growth substrate 104 at operation 356, leaving the fluoropolymer coating layer 106 disposed on the graphene layer 102 functionalized with functional groups 302. The method 300 includes transferring the graphene layer 102 functionalized with functional groups 302 having the fluoropolymer coating layer 106 disposed thereon onto a transfer substrate 108 at operation 358. The method 300 includes removing the fluoropolymer coating layer 106 at operation 360, leaving the functionalized graphene sensor element 310 having a graphene layer 102 functionalized with functional groups 302 disposed on the surface of a transfer substrate 108. In various embodiments, the method 300 further can include sterilizing the graphene assembly, including the graphene functionalized with functional groups and fluoropolymer coating layers disposed on the transfer substrate, as obtained at operation 358, before the step of removing the fluoropolymer coating layer.

### Fluoropolymers

Various embodiments herein include one or more fluoropolymers for use in a fluoropolymer coating layer. Further details about the fluoropolymers are provided as follows. However, it will be appreciated that this is merely provided by way of example and that further variations are contemplated herein.

The fluoropolymer coating layers herein can include one or more fluoropolymers including, but not limited to, perfluoropolymers and perfluoropolyethers. In various embodiments, the perfluoropolymers include amorphous perfluoropolymers. The fluoropolymers suitable for use herein are soluble in various fluorous solvents, examples of which are described further below.

The fluoropolymers suitable for use herein have a solubility in a fluorous solvent where the minimum solubility that can be greater than or equal to 0.05 wt. %, 0.1 wt. %, 0.2 wt. %, 0.3 wt. %, 0.4 wt. %, 0.5 wt. %, 0.6 wt. %, 0.7 wt. %, 0.8 wt. %, 0.9 wt. %, 1.0 wt. %, 2 wt. %, 3 wt. %, 4 wt. %, 5 wt. %, 6 wt. %, 7 wt. %, 8 wt. %, 9 wt. %, or 10 wt. %. In various embodiments, the minimum solubility of the fluoropolymers suitable for use herein can be greater than 10 wt. %. In various embodiments, the fluoropolymers suitable for use herein can have a functional solubility in a fluorous solvent of greater than or equal to 0.1 wt. %.

The fluoropolymers can be applied to the graphene layers using a spin coating process. In various embodiments the fluoropolymers can be applied to the graphene layers using a chemical vapor deposition process, a plasma activated chemical vapor deposition process, a drop coating process, a chemical printing process, and the like.

In various embodiments, the fluoropolymers can be applied to the graphene layers using a spin coating process where the spinning speed includes those that are greater than or equal to 100 revolutions per minute (rpm), 200 rpm, 300 rpm, 400 rpm, 500 rpm, 600 rpm, 700 rpm, 800 rpm, 900 rpm, 1000 rpm, 1100 rpm, 1200 rpm, 1300 rpm, 1400 rpm, 1500 rpm, 1600 rpm, 1700 rpm, 1800 rpm, 1900 rpm, 2000 rpm, 2100 rpm, 2200 rpm, 2300 rpm, 2400 rpm, 2500 rpm, 2600 rpm, 2700 rpm, 2800 rpm, 2900 rpm, or 3000 rpm, or can be an amount falling within a range between any of the foregoing. In various embodiments, the fluoropolymers can be applied to the graphene layers using a spin coating process where the spinning speed is greater than 3000 rpm.

Fluoropolymers herein can be deposited using a spin coating process that utilizes a solvent having a boiling point below 200 degrees Celsius. In various embodiments, fluoropolymers herein can be deposited using a spin coating process that utilizes a solvent having a boiling point below 150 degrees Celsius. In yet other embodiments, fluoropolymers herein can be deposited using a spin coating process that utilizes a solvent having a boiling point below 100 degrees Celsius.

Exemplary fluoropolymers can include, but are not to be limited to, TEFLON™-AF (The Chemours Co., Wilmington, Del., USA), CYTOP™ (Asahi Glass Co., Ltd., Chiyoda, Tokyo, Japan), Hyflon™ AD (Solvay Group, Neder-Over-Heembeek, Brussels, Belgium), and Krytox™ (The Chemours Co., Wilmington, Del., USA). Chemical structures of some exemplary fluoropolymers are presented in Table 1 below. Additional fluoropolymers can include poly[oxy(1,1,2,2,3,3-hexafluoro-1,2-propanediyl)] and poly[oxy(1,1,2,2,3,3-hexafluoro-1,3-propanediyl)], or derivatives thereof.

TABLE 1
Exemplary Fluoropolymers

| | | |
|---|---|---|
| (A) | 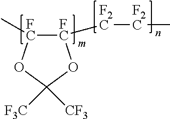 | Teflon ™-AF (The Chemours Co., Wilmington, Delaware, USA) |
| (B) | 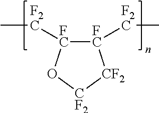 | CYTOP ™ (Asahi Glass Co., Ltd., Chiyoda, Tokyo, Japan) |
| (C) | 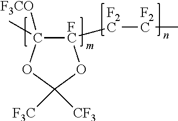 | Hyflon ™ AD (Solvay Group, Neder- Over-Heembeek, Brussels, Belgium) |
| (D) | 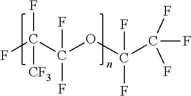 | Krytox ™ (The Chemours Co., Wilmington, Delaware, USA) |

In various embodiments, the fluoropolymer suitable for use herein can include fluoroethylenes such as poly[4,5-difluoro-2,2-bis(trifluoromethyl)-1,3-dioxole-co-tetrafluoroethylene] (i.e., Teflon™-AF, The Chemours Co., Wilmington, Del., USA), or derivatives thereof. Suitable poly[4,5-difluoro-2,2-bis(trifluoromethyl)-1,3-dioxole-co-tetrafluoroethylene] can include those where the mole ratio of dioxole to tetrafluoroethylene is from 1:99 to 99:1. In various embodiments, suitable poly[4,5-difluoro-2,2-bis(trifluoromethyl)-1,3-dioxole-co-tetrafluoroethylene] can include those where the mole ratio of dioxole to tetrafluoroethylene is from 1:50 to 50:1. In other embodiments, suitable poly[4,5-difluoro-2,2-bis(trifluoromethyl)-1,3-dioxole-co-tetrafluoroethylene] can include those where the mole ratio of dioxole to tetrafluoroethylene is from 1:25 to 25:1. In yet other embodiments, suitable poly[4,5-difluoro-2,2-bis(trifluoromethyl)-1,3-dioxole-co-tetrafluoroethylene] can include those where the mole ratio of dioxole to tetrafluoroethylene is from 1:5 to 5:1.

The fluoropolymer coating layer can include those having a thickness of from 10 nanometers (nm) to 300 nm. In some embodiments, the thickness can be greater than or equal to 10 nm, 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, or 300 nm, or can be an amount falling within a range between any of the foregoing. In various embodiments, the fluoropolymer coating layer is at least about 10 nanometers thick. In various embodiments, the fluoropolymer coating layer is at least about 20 nanometers thick. In various embodiments, the fluoropolymer coating layer is at least about 100 nanometers thick. In various embodiments, the fluoropolymer coating layer is at least about 200 nanometers thick.

### Fluorous Solvents

Various embodiments herein include one or more fluorous solvents. Further details about the fluorous solvents are provided as follows. As used herein, the term “fluorous solvent” refers to a solvent containing multiple fluorine atoms in place of hydrogen atoms in analogous hydrocarbon-based solvents. However, it will be appreciated that this is merely provided by way of example and that further variations are contemplated herein.

The solvents herein can include those selected from the groups including perfluoroalkanes, partially fluorinated alkanes, partially fluorinated haloalkanes, perfluorinated mono- or polycyclic alkanes, perfluorinated singly or multiply alkyl substituted mono- or polycyclic alkane, perfluoroaromatics, (perfluoroalkyl)benzenes, perfluoroethers, perfluorodiethers, perfluorotriethers, perfluoroalkyl alkyl ethers, perfluoro(trialkylamines), or mixtures of two or more of any preceding solvents.

The solvents can specifically include various straight chain and branched perfluoroalkanes including perfluorohexane, perfluoroheptane, perfluorooctane (also referred to as PF5080™, 3M, Maplewood, Minn., USA) perfluoronane; various straight chain, branched and cyclic partially fluorinated alkanes, such as 2H-3H-decafluoropentane and 1,1,1,3,3-pentafluorobutane; various straight chain, branched and cyclic partially fluorinated haloalkanes, such as 1,1-dichloro-2,2,3,3,3-pentafluoropropane; various perfluorinated mono- or polycyclic alkanes, and perfluorinated singly or multiply alkylsubstituted mono- or polycyclic alkanes, such as perfluorocyclohexane, octadecafluorodecahydronaphthalene, perfluoro(methylcyclohexane), perfluoro(dimethylcyclohexane), and perfluoro(methyldecalin); various perfluoroaromatics, such as hexafluorobenzene; various (perfluoroalkyl)benzenes, such as trifluoromethylbenzene (also referred to as trifluorotoluene); various perfluoroethers, perfluorodiethers, and perfluorotriethers, such as perfluoro(diethyl ether) and compounds with one or multiple branching points, such as perfluoro(diisopropyl ether); various perfluoroalkyl alkyl ethers, such as nonafluorobutyl methyl ether and nonafluorobutyl ethyl ether, and perfluoroalkyl alkyl ethers in which either the perfluoroalkyl or the alkyl substituent or both are branched, such as perfluoro(2 -methylpropyl) methyl ether; perfluoro(trialkylamines) such as perfluoro(tributylamine); or any mixtures of two or more of any of these solvents. In various embodiments the solvent can include Novec™ 7100 Engineered Fluid (3M, Maplewood, Minn., USA). In various embodiments, some exemplary fluorous solvents can include C2 to C10 fluorous solvents.

Exemplary fluorous solvents and their chemical structures are listed below in Table 2.

TABLE 2
Exemplary Fluorous Solvents

| | | |
|---|---|---|
| (A) | 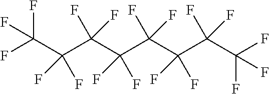 [SMILES: FC(F)(F)C(F)(F)C(F)(F)C(F)(F)C(F)(F)C(F)(F)C(F)(F)C(F)(F)F] | perfluorooctane (i.e., PF5080 ™) |
| (B) | 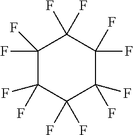 [SMILES: FC1(F)C(F)(F)C(F)(F)C(F)(F)C(F)(F)C1(F)F] | perfluorocyclohexane |
| (C) | 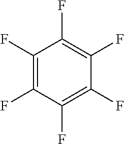 [SMILES: Fc1c(F)c(F)c(F)c(F)c1F] | perfluorobenzene |
| (D) | 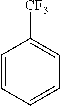 | trifluoromethyl- benzene |
| (E) | 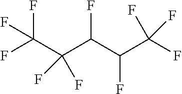 [SMILES: FC(C(F)C(F)(F)C(F)(F)F)C(F)(F)F] | 2H,3H- decafluoropentane |
| (F) | 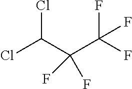 | 1,1-dichloro-2,2,3,3,3- pentafluoropropane |
| (G) | 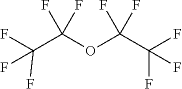 [SMILES: FC(F)(F)C(F)(F)OC(F)(F)C(F)(F)F] | perfluoro(diethyl ether) |
| (H) | 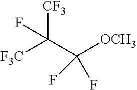 [SMILES: COC(F)(F)C(F)(C(F)(F)F)C(F)(F)F] | perfluoro(2- methylpropyl) methyl ether |

The fluorous solvents can include those having a boiling point below 200 degrees Celsius. In some embodiments, fluorous solvents can include those having a boiling point below 150 degrees Celsius. In other embodiments, fluorous solvents can include those having a boiling point below 100 degrees Celsius. In some embodiments, the boiling point can be less than or equal to 250° C., 240° C., 230° C., 220° C., 210° C., 200° C., 190° C., 180° C., 170° C., 160° C., 150° C., 140° C., 130° C., 120° C., 110° C., 100° C., 90° C., 80° C., 70° C., 60° C., or 50° C., or can be an amount falling within a range between any of the foregoing.

### Graphene Sensor Element

Various embodiments herein include a graphene sensor element. Further details about the graphene sensor element are provided as follows. However, it will be appreciated that this is merely provided by way of example and that further variations are contemplated herein.

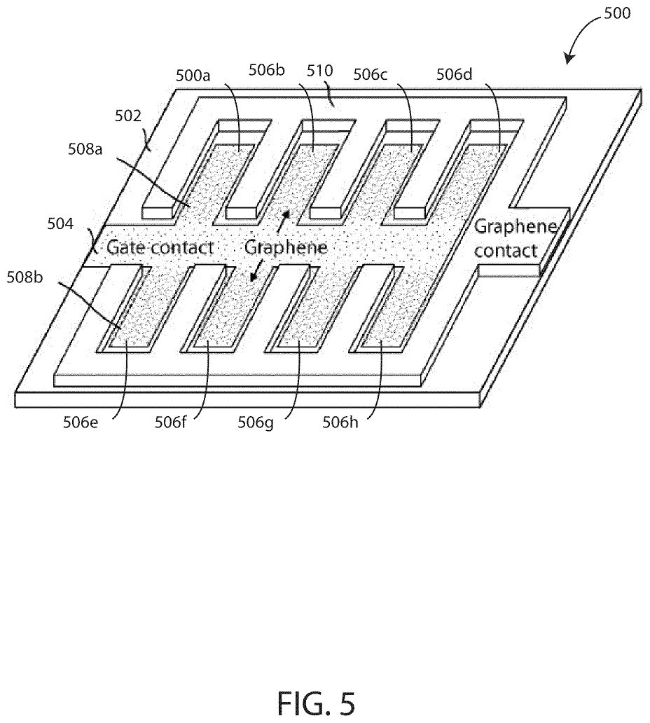

A graphene sensor element is included having a graphene layer and a fluoropolymer coating layer over the graphene layer. In various embodiments, the graphene sensor elements herein can include graphene-based variable capacitors (or graphene varactors). However, in some embodiments, the graphene sensor elements herein can be formed with other materials such as borophene. Referring now to FIG. 5, a schematic view of a graphene varactor 500 is shown in accordance with the embodiments herein. It will be appreciated that graphene varactors can be prepared in various ways with various geometries, and that the graphene varactor shown in FIG. 5 is just one example in accordance with the embodiments herein.

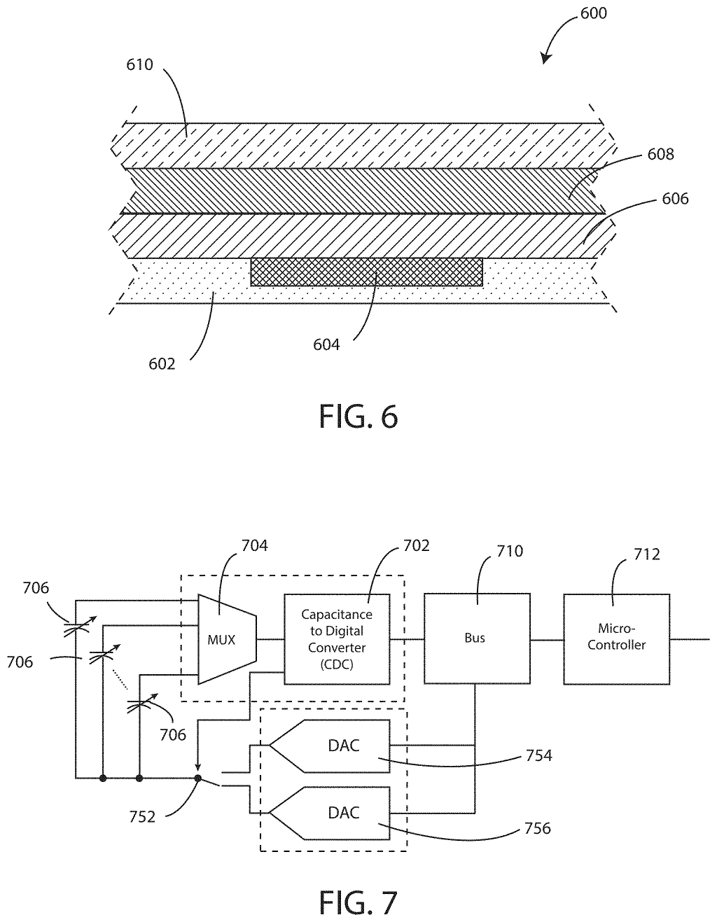

Each graphene varactor 500 can include an insulator layer 502, a gate electrode 504 (or “gate contact”), a dielectric layer (not shown in FIG. 5), one or more graphene layers, such as graphene layers 508a and 508b, and a contact electrode 510 (or “graphene contact”). In some embodiments, the graphene layer(s) 508a-b can be contiguous, while in other embodiments the graphene layer(s) 508a-b can be non-contiguous. Gate electrode 504 can be deposited within one or more depressions formed in insulator layer 502. Insulator layer 502 can be formed from an insulative material such as silicon dioxide, formed on a silicon substrate (wafer), and the like. Gate electrode 504 can be formed by an electrically conductive material such as chromium, copper, gold, silver, tungsten, aluminum, titanium, palladium, platinum, iridium, nickel, and any combinations or alloys thereof, which can be deposited on top of or embedded within the insulator layer 502. The dielectric layer can be disposed on a surface of the insulator layer 502 and the gate electrode 504. The graphene layer(s) 508a-b can be disposed on the dielectric layer. The dielectric layer will be discussed in more detail below in reference to FIG. 6.

Each graphene varactor 500 can include eight gate electrode fingers 506a-506h. It will be appreciated that while graphene varactor 500 shows eight gate electrode fingers 506a-506h, any number of gate electrode finger configurations can be contemplated. In some embodiments, an individual graphene varactor can include fewer than eight gate electrode fingers. In some embodiments, an individual graphene varactor can include more than eight gate electrode fingers. In other embodiments, an individual graphene varactor can include two gate electrode fingers. In some embodiments, an individual graphene varactor can include 1, 2, 3, 4, 5, 6, 7, 8, 9, 10, or more gate electrode fingers.

Each graphene varactor 500 can include one or more contact electrodes 510 disposed on portions of the graphene layers 508a and 508b. Contact electrode 510 can be formed from an electrically conductive material such as chromium, copper, gold, silver, tungsten, aluminum, titanium, palladium, platinum, iridium, nickel, and any combinations or alloys thereof. Further aspects of exemplary graphene varactors can be found in U.S. Pat. No. 9,513,244, the content of which is herein incorporated by reference in its entirety.

Referring now to FIG. 6, a schematic cross-sectional view of a portion of a graphene varactor 600 is shown in accordance with various embodiments herein. The graphene varactor 600 can include an insulator layer 602 and a gate electrode 604 recessed into the insulator layer 602. The gate electrode 604 can be formed by depositing an electrically conductive material in the depression in the insulator layer 602, as discussed above in reference to FIG. 5. A dielectric layer 606 can be formed on a surface of the insulator layer 602 and the gate electrode 604. The dielectric layer 606 can include the transfer substrate, as discussed elsewhere herein. In some examples, the dielectric layer 606 can be formed of a material, such as, silicon dioxide, silicon oxide, aluminum oxide, hafnium dioxide, hafnium oxide, zirconium dioxide, zirconium oxide, hafnium silicate, or zirconium silicate.

The graphene varactor 600 can include a single graphene layer 608 that can be disposed on a surface of the dielectric layer 606. The graphene layer 608 can be surface-modified with a modification layer 610. In various embodiments, the modification layer can include one or more functional groups, as discussed further below. It will be appreciated that in some embodiments, the graphene layer 608 is not surface-modified.

During use of the graphene varactors as described herein, a sweep performed on the excitation voltage of an entire gas measurement system provides data regarding the Dirac point (the voltage when the capacitance is at a minimum). As analytes are sensed by the graphene varactors, the voltage of the Dirac point can shift to a higher or lower value. The shape of the curve can also change. The changes in the sweep curve can be used as sensing features that can be attributed to the graphene varactor's response to the analyte/receptor interaction. Employing a fast sampling system while sweeping the voltage can provide kinetic information. Thus, the complete response can be measured at steady state, which can provide data related to how long it took to get to steady state (kinetic information).

The gas sensing systems described herein can include circuitry for generating signals from the graphene varactors. Such circuitry can include active and passive sensing circuits. Such circuitry can implement using wired (direct electrical contact) or wireless sensing techniques.

Referring now to FIG. 7, a schematic diagram is shown of circuitry to measure the capacitance of a plurality of graphene sensor elements in accordance with another embodiment herein. The circuitry can include a capacitance to digital converter (CDC) 702 in electrical communication with a multiplexor 704. The multiplexor 704 can provide selective electrical communication with a plurality of graphene varactors 706. The connection to the other side of the graphene varactors 706 can be controlled by a switch 752 (as controlled by the CDC) and can provide selective electrical communication with a first digital to analog converter (DAC) 754 and a second digital to analog converter (DAC) 756. The other side of the DACs 754, 756 can be connected to a bus device 710, or in some cases, the CDC 702. In some embodiments, the bus device 710 can interface with a microcontroller 712 or other computing device.

In this case, the excitation signal from the CDC controls the switch between the output voltages of the two programmable Digital to Analog Converters (DACs). The programmed voltage difference between the DACs determines the excitation amplitude, providing an additional programmable scale factor to the measurement and allowing measurement of a wider range of capacitances than specified by the CDC. The bias voltage at which the capacitance is measured is equal to the difference between the bias voltage at the CDC input (via the multiplexor, usually equal to VCC/2, where VCC is the supply voltage) and the average voltage of the excitation signal, which is programmable. In some embodiments, buffer amplifiers and/or bypass capacitance can be used at the DAC outputs to maintain stable voltages during switching. Many different ranges of DC bias voltages can be used. In some embodiments, the range of DC bias voltages can be from −3 V to 3 V, or from −1 V to 1 V, or from −0.5 V to 0.5 V. Further aspects of exemplary sensing circuitry is provided in U.S. Publ. Pat. Appl. No. 2019/0025237, the content of which is herein incorporated by reference in its entirety.

### Functionalization Groups

Various embodiments herein include functionalization groups disposed on the graphene layers described. Further details about exemplary functionalization groups are provided as follows. However, it will be appreciated that this is merely provided by way of example and that further variations are contemplated herein.

The graphene sensor elements described herein can include those in which a graphene layer has been surface-modified through non-covalent π-π stacking interactions between graphene and π-electron-rich molecules, such as, for example, pyrene, pyrene derivatives, and other compounds with aryl groups. The graphene sensor elements described herein can alternatively include those in which a graphene layer has been surface-modified through non-covalent electrostatic interactions between graphene and molecules with C1-C20 alkyl chains or molecules with multiple C1-C20 alkyl groups. Additional functionalization groups can be suitable for use herein as provided in U.S. Pat. App. Publ. No. 2019/0257825 A1; U.S. application Ser. No. 16/393,177; and U.S. App. Ser. No. 62/889,387 the contents of which are herein incorporated by reference in their entirety. In some embodiments, the graphene sensor elements described herein can include those in which a graphene layer has been surface-modified through covalently bonded functionalization groups.

Aspects may be better understood with reference to the following examples. These examples are intended to be representative of specific embodiments, but are not intended as limiting the overall scope of embodiments herein.

### EXAMPLES

### Example 1: Polymethylmethacrylate Transfer of Graphene

Graphene monolayers were grown on a copper substrate to yield a graphene assembly including a single graphene layer disposed on a surface of a copper substrate layer. A polymethylmethacrylate (PMMA) polymer layer was spin-coated onto the surface of the graphene layer and the copper substrate layer was removed using the etchant ammonium persulfate. The graphene layer was then transferred to a silicon dioxide substrate and the PMMA was dissolved with strong solvent for up to 48 hours at 40° C. under agitation using a magnetic stir bar set to 500 rpm. to leave behind the graphene layer disposed on the silicon dioxide substrate.

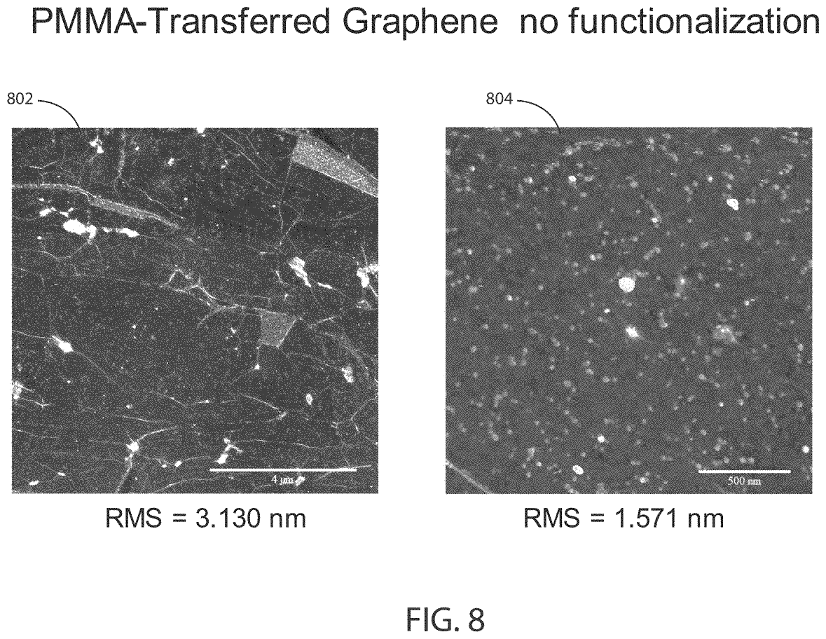

Atomic force microscopy imaging was performed to detect surface roughness of the surface of the PMMA-transferred graphene having no functionalization. The results of the AFM imaging of the PMMA-transferred graphene having no functionalization are shown in FIG. 8. AFM imaging can measure the dimensions of a surface and can detect surface roughness due to processing and preparation. One measure of surface height deviation from an average plane of a surface is the root mean square (RMS). A PMMA-transferred graphene layer at having no functionalization is shown in 802 at 4 micron (μm) magnification. The image shows a PMMA-transferred graphene layer having various regions of PMMA residue left intact on the surface of the graphene layer (as shown by bright spots and/or streaks), and having a root mean square (RMS) of 3.130 nm. A separate PMMA-transferred graphene layer having no functionalization is shown in 804 at 500 nanometer (nm) magnification, having various regions of residue left intact on the surface of the graphene layer and an RMS of 1.571 nm.

### Example 2: Fluoropolymer Transfer of Graphene

Graphene monolayers were grown on a copper substrate to yield a graphene assembly including a single graphene layer disposed on a surface of a copper substrate layer. A 1 wt. % solution of Teflon™ AF 1600 was prepared in the solvent PF5080™ The solution was spin-coated onto the surface of the graphene layer to create a layer of Teflon™ AF 1600, and the solvent was evaporated. The copper substrate layer was removed using the etchant ferric chloride. The graphene layer with a Teflon™ AF 1600 disposed thereon was then transferred to a silicon dioxide substrate. The Teflon™ AF 1600 layer was immersed in a bath of the fluorous solvent Novec™ 7100 for up to 48 hours at 40° C. under agitation using a magnetic stir bar set to 500 rpm. The Novec™ 7100 was changed every 12 hours. The Novec™ 7100 dissolved the Teflon™ AF 1600 layer to leave behind the graphene layer disposed on the silicon dioxide substrate.

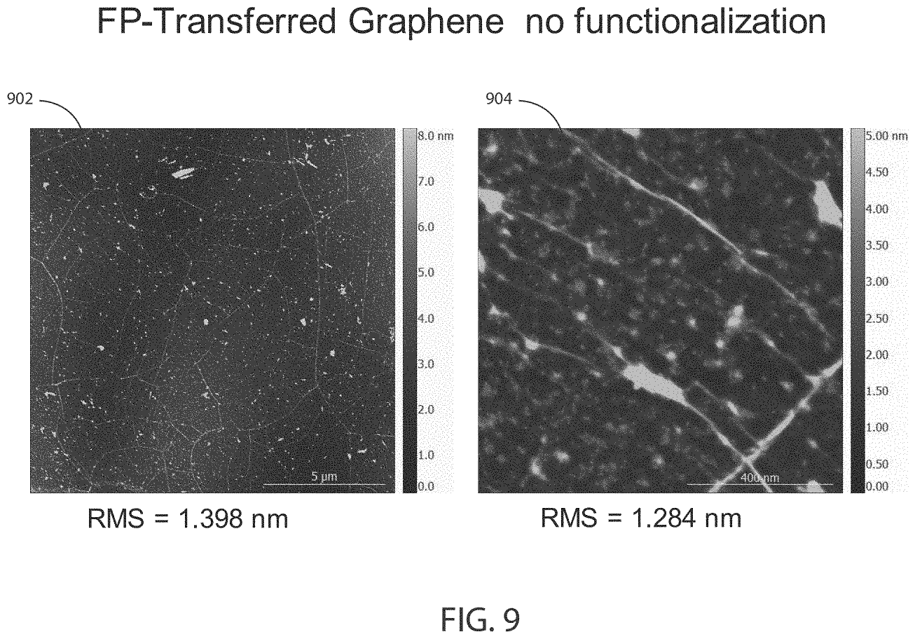

Atomic force microscopy imaging was performed to detect surface roughness of the surface of the fluoropolymer-transferred graphene having no functionalization. The results of the AFM imaging of the Teflon™ AF 1600-transferred graphene having no functionalization are shown in FIG. 9. The Teflon™ AF 1600-transferred graphene layer having no functionalization is shown in 902 at 5 μm magnification. The image shows a Teflon™ AF 1600-transferred graphene having various regions of fluoropolymer (FP) residue left intact on the surface of the graphene layer (as shown by bright spots and/or streaks), and having a root mean square (RMS) of 1.398 nm. A separate Teflon™ AF 1600-transferred graphene layer having no functionalization is shown in 904 at 400 nm magnification having various small regions of residue left intact on the surface of the graphene layer, and having an RMS of 1.284 nm. As such, the fluoropolymer transfer process with a fluorous solvent was superior to the PMMA transfer process illustrated previously and, specifically, left substantially less residue behind.

### Example 3: Fluoropolymer Transfer of Graphene Functionalized with Pyrene-CH₂COOCH₃

Graphene monolayers were grown on a copper substrate to yield a graphene assembly including a single graphene layer disposed on a surface of a copper substrate layer. The graphene layer was functionalized with the it-rich molecule pyrene-CH₂COOCH₃ (pyr-CH₂COOCH₃). A fluoropolymer layer was spin-coated onto the surface of the graphene layer and the copper substrate layer was removed using the etchant ferric chloride. The graphene layer was then transferred to a silicon dioxide substrate and the fluoropolymer was dissolved with a fluorous solvent for up to 48 hours at 40° C. under agitation using a magnetic stir bar set to 500 rpm to leave behind the graphene layer disposed on the silicon dioxide substrate.

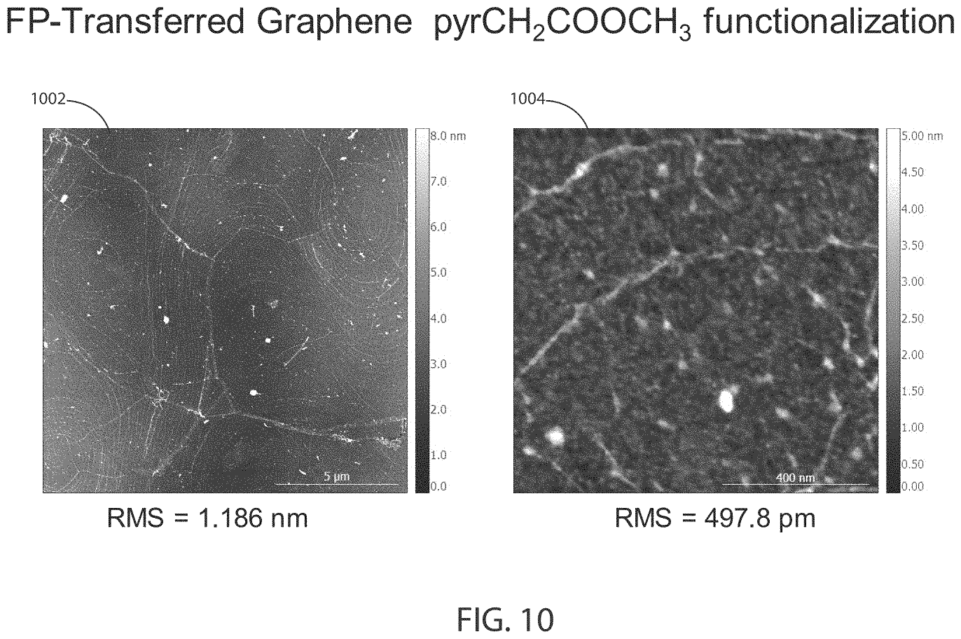

Atomic force microscopy imaging was performed to detect surface roughness of the surface of the fluoropolymer-transferred graphene functionalized with pyr-CH₂COOCH₃. The results of the AFM imaging of the fluoropolymer-transferred graphene functionalized with pyr-CH₂COOCH₃ are shown in FIG. 10. A fluoropolymer-transferred graphene layer functionalized with pyr-CH₂COOCH₃ is shown in 1002 at 5 μm magnification. The image shows a fluoropolymer-transferred graphene functionalized with pyr-CH₂COOCH₃ having various regions of FP residue left intact on the surface of the graphene layer (as shown by bright spots and/or streaks), and having a root mean square (RMS) of 1.186 nm. A separate fluoropolymer-transferred graphene functionalized with pyr-CH₂COOCH₃ having no functionalization is shown in 1004 at 400 nm magnification having various small regions of fluoropolymer residue left intact on the surface of the graphene layer, and having an RMS of 497.8 picometers (μm). As such, the fluoropolymer transfer process with a fluorous solvent was superior to the PMMA transfer process illustrated previously and, specifically, left substantially less residue behind.

### Example 4: Comparative Transfer of Non-Functionalized Graphene With PMMA and Various Etchants

Single graphene monolayers were grown on multiple copper substrates. A polymethylmethacrylate (PMMA) polymer layer was spin-coated onto the surface of each graphene layer and the copper substrate layer was removed using either ammonium persulfate or ferric chloride. Each graphene layer was then transferred to a separate silicon dioxide substrate. The PMMA was dissolved with a strong solvent, and the dissolution of the PMMA layer left behind a non-functionalized graphene layer disposed on a silicon dioxide substrate.

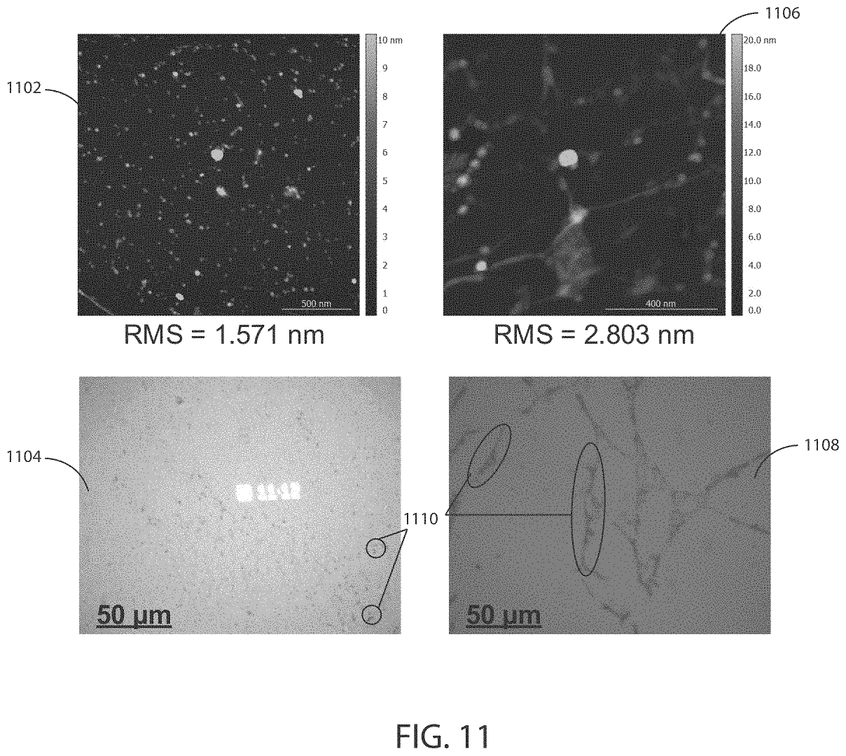

Atomic force microscopy imaging and optical imaging was performed to detect surface roughness of the surface of the PMMA-transferred graphene (non-functionalized). The results of the AFM imaging and optical imaging of the PMMA-transferred graphene (non-functionalized) are shown in FIG. 11. PMMA-transferred graphene (non-functionalized), where the copper substrate has been removed using ammonium persulfate, is shown in 1102 (AFM image at 500 nm magnification; RMS 1.517 nm) and 1104 (optical image at 50 μm magnification). The AFM and optical images reveal some regions of PMMA residue 1110 on the surface of the graphene as seen in optical image 1104. PMMA-transferred graphene (non-functionalized), where the copper substrate has been removed using ferric chloride, is shown in 1106 (AFM image at 400 nm magnification; RMS 2.803 nm) and 1108 (optical image at 50 μm magnification). The AFM and optical images reveal a significantly larger regions of PMMA residue 1110 on the surface of the graphene as seen in 1108. Without wishing to be bound by any particular theory, it is believed that the solvent ferric chloride increases cross-linking of the PMMA and thus increases the amount residue left behind on the surface of the PMMA-transferred graphene layer when compared to ammonium persulfate.

### Example 5: Transfer of Non-Functionalized Graphene and Pyr-CH₂COOCH₃-Functionalized Graphene Using Fluoropolymer

Single graphene monolayers were grown on multiple copper substrates. Half of the graphene monolayers were functionalized with Pyr-CH₂COOCH₃. A fluoropolymer layer was spin-coated onto the surface of each graphene layer and the copper substrate layer was removed using ferric chloride. Each graphene layer was then transferred to a separate silicon dioxide substrate. The fluoropolymer was dissolved with a fluorous solvent for up to 48 hours at 40° C. under agitation using a magnetic stir bar set to 500 rpm, and the dissolution of the fluoropolymer layer left behind a graphene layer disposed on a silicon dioxide substrate.

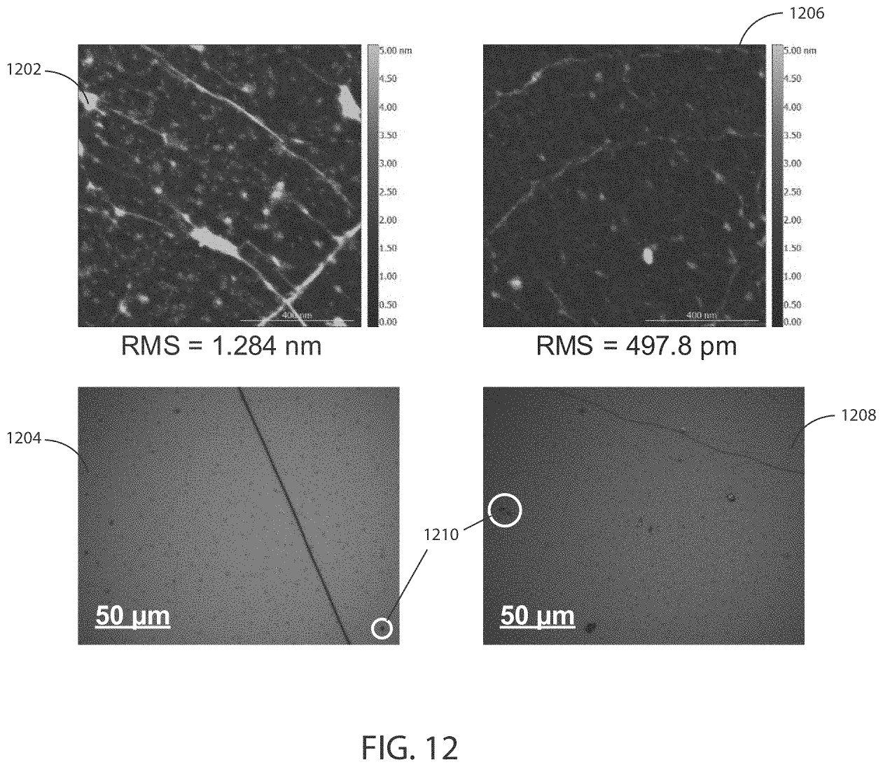

Atomic force microscopy imaging and optical imaging was performed to detect surface roughness of the surface of the fluoropolymer-transferred graphene non-functionalized or functionalized with Pyr-CH₂COOCH₃. The results of the AFM imaging and optical imaging of the non-functionalized fluoropolymer-transferred graphene or fluoropolymer-transferred graphene functionalized with Pyr-CH₂COOCH₃ are shown in FIG. 12. Fluoropolymer-transferred graphene that was not functionalized and where the copper substrate has been removed using ferric chloride is shown in 1202 (AFM image at 400 nm magnification; RMS 1.284 nm) and 1204 (optical image at 50 μm magnification). The AFM and optical images of non-functionalized fluoropolymer-transferred graphene reveal some regions of residue 1210 on the surface of the graphene layer (as shown by bright spots and/or streaks) on the surface of the graphene. Fluoropolymer-transferred graphene that was functionalized with Pyr-CH₂COOCH₃ where the copper substrate has been removed using ferric chloride is shown in 1206 (AFM image at 400 nm magnification; RMS 497.8 picometers (μm)) and 1208 (optical image at 50 μm magnification). The AFM and optical images of fluoropolymer-transferred graphene functionalized with Pyr-CH₂COOCH₃ reveal significantly fewer regions of residue 1210 on the surface of the graphene. As such, the fluoropolymer transfer process with a fluorous solvent was superior to the PMMA transfer process illustrated previously and, specifically, left substantially less residue behind.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A method of producing a graphene sensor element comprising:
forming a graphene layer on a growth substrate;
applying a fluoropolymer coating layer over the graphene layer;
removing the growth substrate;
transferring the graphene and fluoropolymer coating layers onto a transfer substrate such that the graphene layer is disposed on the transfer substrate and the fluoropolymer layer is disposed on the graphene layer; and
removing the fluoropolymer coating layer.

2. The method of claim 2, the growth substrate comprising copper.

3. The method of claim 2, the fluoropolymer comprising poly[4,5-difluoro-2,2-bis(trifluoromethyl)-1,3-dioxole-co-tetrafluoroethylene], or derivatives thereof.

4. The method of claim 3, wherein a mole ratio of dioxole to tetrafluoroethylene is from 1:99 to 99:1.

5. The method of claim 2, the fluoropolymer comprising poly[oxy(1,1,2,2,3,3-hexafluoro-1,2-propanediyl)], poly[oxy(1,1,2,2,3,3-hexafluoro-1,3-propanediyl)], or derivatives thereof.

6. The method of claim 2, wherein the fluoropolymer has a solubility in a solvent of greater than 0.1 wt. %.

7. The method of claim 2, wherein applying a fluoropolymer comprises a spin coating process, an ink-jet printing, a spray coating process, or a chemical vapor deposition process.

8. The method of claim 2, wherein removing the growth substrate comprises applying a ferric chloride solution or an ammonium persulfate solution.

9. The method of claim 2, wherein the fluoropolymer coating layer is at least about 10 nanometers thick.

10. The method of claim 2, further comprising sterilizing the graphene and fluoropolymer coating layers disposed on the transfer substrate before removing the fluoropolymer coating layer.

11. The method of claim 2, wherein removing the fluoropolymer coating layer comprises applying a solvent comprising a perfluoroalkane, a partially fluorinated alkane, a partially fluorinated haloalkane, a perfluorinated mono- or polycyclic alkane, a perfluorinated singly or multiply alkyl substituted mono- or polycyclic alkane, a perfluoroaromatic, a (perfluoroalkyl)benzene, a perfluoroether, a perfluorodiether, a perfluorotriether, a perfluoroalkyl alkyl ether, a perfluoro(trialkylamine), or mixtures of two or more of any preceding solvents.

12. The method of claim 2, wherein removing the fluoropolymer coating layer is performed immediately before use of the graphene sensor element to analyze a gas sample.

13. A method of producing a graphene sensor element comprising:
forming a graphene layer on a growth substrate;
functionalizing the graphene layer;
applying a fluoropolymer coating layer over the graphene layer;
removing the growth substrate;
transferring the graphene and fluoropolymer coating layers onto a transfer substrate; and
removing the fluoropolymer coating layer.

14. The method of claim 13, the growth substrate comprising copper.

15. The method of claim 13, the fluoropolymer coating layer comprising one or more fluoropolymers comprising perfluoropolymers and perfluoropolyethers.

16. The method of claim 13, the fluoropolymer comprising poly[4,5-difluoro-2,2-bis(trifluoromethyl)-1,3-dioxole-co-tetrafluoroethylene]; poly [oxy(1,1,2,2,3,3-hexafluoro-1,2-propanediyl)]; or poly[oxy(1,1,2,2,3,3-hexafluoro-1,3-propanediyl)]; or derivatives thereof.

17. The method of claim 13, wherein removing the growth substrate comprises applying a ferric chloride solution or an ammonium persulfate solution.

18. The method of claim 13, further comprising sterilizing the graphene sensor element before removing the fluoropolymer coating layer.

19. The method of claim 13, wherein removing the fluoropolymer coating layer comprises applying a solvent comprising a perfluoroalkane, a partially fluorinated alkane, a partially fluorinated haloalkane, a perfluorinated mono- or polycyclic alkane, a perfluorinated singly or multiply alkyl substituted mono- or polycyclic alkane, a perfluoroaromatic, a (perfluoroalkyl)benzene, a perfluoroether, a perfluorodiether, a perfluorotriether, a perfluoroalkyl alkyl ether, a perfluoro(trialkylamine), or mixtures of two or more of any preceding solvents.

20. The method of claim 13, wherein removing the fluoropolymer coating layer is performed immediately before use of the graphene sensor element to analyze a gas sample.
